(19) United States
(12) Patent Application Publication
(10) Pub. No.: US 2025/0135239 A1
(43) Pub. Date: May 1, 2025

(54) METHOD FOR REDUCING FOREIGN BODY RESPONSE FROM NEURAL IMPLANTS

(71) Applicant: Actuated Medical, Inc., Bellefonte, PA (US)

(72) Inventors: Alanoud S Alsubhi, State College, PA (US); Roger B Bagwell, Bellefonte, PA (US); Jenna K Greaser, State College, PA (US); Kevin A Snook, State College, PA (US); Natasha A Tirko, State College, PA (US); Kyle W Gheres, Pennsylvania Furnace, PA (US); Ryan S Clement, State College, PA (US)

(73) Assignee: Actuated Medical, Inc., Bellefonte, PA (US)

(21) Appl. No.: 19/009,861

(22) Filed: Jan. 3, 2025

Related U.S. Application Data
(62) Division of application No. 17/837,766, filed on Jun. 10, 2022.
(60) Provisional application No. 63/231,410, filed on Aug. 10, 2021.

(51) Int. Cl. A61N 7/00; A61N 1/05; B06B 1/06

(52) U.S. Cl. CPC A61N 7/00; A61N 1/0551; B06B 1/0655; A61N 2007/0004; B06B 2201/76

(57) ABSTRACT

A method for reducing foreign body response in a subject caused by an electrode implanted in a subject's neural tissue at a target site provides positioning a device having a body, ultrasonic transducer, and acoustic coupling medium in contact with the neural tissue proximate to the target site. The method continues with activating a transducer, generating acoustic vibrations, transmitting the acoustic vibrations to the neural tissue at the target site, and applying the acoustic vibrations to the neural tissue at the target site sufficient to reduce the foreign body response in the neural tissue at the target site. The acoustic vibrations are preferably in the ultrasonic range and are pulsed for a duration of 5-200 milliseconds. A treatment protocol may consist of repeat sessions in which the transducer is activated and the acoustic vibrations are applied for 1-15 minutes, then turned off for 1-15 minutes, and repeated 2-10 times.

### CLAIM OF PRIORITY

The present application is a Divisional of co-pending application Ser. No. 17/837,766, filed on Jun. 10, 2022, which claims the benefit of U.S. Provisional Application Ser. No. 63/231,410, filed Aug. 10, 2021, the contents of all of which are incorporated herein by reference in their entireties.

### STATEMENT REGARDING FEDERALLY SPONSORED RESEARCH OR DEVELOPMENT

This invention was made with government support under EB028055 awarded by the National Institutes of Health. The government has certain rights in the invention.

### FIELD OF THE INVENTION

This invention relates to a device for delivering acoustic stimulation to tissue surrounding an implant, reducing bodily response to the implant and improving implant performance.

### BACKGROUND

Implants, such as chronically implanted microelectrode arrays designed to interface with neural tissue, hold great potential for revolutionizing treatment of a range of medical conditions. Applications of neural implants include neural-based control of prosthetic limbs by amputees, brain-machine interfacing for paraplegics, selective ablation and/or inactivation of problematic neural pathways, or control or enhancement of organ function, to name a few. Programs like SPARC, the BRAIN Initiative, and BrainGate are bringing new neuroprosthetic devices to patients, and researchers predict that neural implants will be more widely implemented in humans in the next 10 years. Non-penetrating neural implant electrode arrays such as EEG electrodes and nerve cuffs have seen increased clinical application in recent years, but such systems have limited spatial resolution, making them less ideal for future applications requiring more precise stimulation or recording. Penetrating neural electrode arrays offer significantly improved temporal and spatial resolution but suffer from multiple complications which restrict their clinical use.

The trauma of implantation, including the dimpling of local tissue and nerves, may decrease implant recording yield and can cause and/or accelerate glial scarring which isolates the implant from the target tissue. Chronically placed neural penetrating members that remain resident in tissue cause a reactive tissue response, the foreign body response (FBR), involving astrocytes and microglia that result in the formation of a cellular sheath or scar around the penetrating member. The response is highly complex with various chemical signaling pathways, cell types, and damage involved, but overall involves an initial acute phase of glial scarring in response to the initial injury followed by chronic inflammation. The range of applications of neural implants is expanding. However, poor longevity and variable recording quality are frequently points of failure in implant systems. This isolating glial scarring and neural cell loss occurs within 100-500 um of implant sites.

The FBR limits the clinical potential of chronic neural implants, therefore, minimizing FBR would improve chronic implant performance. Current efforts to minimize FBR include: alteration of array composition and geometry, bio-mimicking coatings, and the creation of floating arrays (i.e., arrays not fixed to the skull) which freely move with the brain; despite these efforts, performance degradation plagues all array types. Bioactive implant coatings or features that can improve host-implant integration and inflammatory mediators such as dexamethasone show short term success, but the long-term effect on neural interface performance after depletion of the bioactive element is unclear.

In one study, implants were engineered to release a brain derived neurotrophic factor (BDNF) analog (Fon D, Zhou K, Ercole F, et al. Nanofibrous scaffolds releasing a small molecule BDNF-mimetic for the re-direction of endogenous neuroblast migration in the brain. Biomaterials. 2014; 35(9): 2692-2712). The BDNF analog increased neurite growth onto implanted scaffolds and the beneficial effect ended when the BDNF supply was exhausted. A healthy, neural-supportive, anti-inflammatory microenvironment around penetrating electrode arrays may be effectuated by the introduction of increased BDNF, along with other neurotrophic factors. Limiting inflammation has been proven to improve electrode interfaces, as shown in a study of caspase-1 knock-out mice (Kozai T K, Li X, Bodily L M, et al. Effects of caspase-1 knockout on chronic neural recording quality and longevity: Insight into cellular and molecular mechanisms of the reactive tissue response, Biomaterials, 2014; 35(36): 9620-9634). BDNF has been shown to block the activity of caspase, an enzyme involved in cell death; BDNF at the electrode site may reduce inflammation in a similar way.

Transcranial ultrasound stimulation, such as low-intensity pulsed ultrasound (LIPUS), has been reported to improve behavioral and/or histological outcomes in preclinical models of experimental traumatic brain injury (TBI) and stroke (Su W S, Wu C H, Chen S F, Yang F Y. Transcranial ultrasound stimulation promotes brain-derived neurotrophic factor and reduces apoptosis in a mouse model of traumatic brain injury. Brain Stimul. 2017; 10(6): 1032-1041); (Chen S F, Su W S, Wu CH, Lan T H, Yang F Y. Transcranial Ultrasound Stimulation Improves Long-Term Functional Outcomes and Protects Against Brain Damage in Traumatic Brain Injury. Mol Neurobiol. 2018; 55(8): 7079-7089); (Lin W T, Chen R C, Lu W W, Liu S H, Yang F Y. Protective effects of low-intensity pulsed ultrasound on aluminum-induced cerebral damage in Alzheimer's disease rat model. Sci Rep. 2015; 5). The protective effects of transcranial therapeutic ultrasound are likely caused at least partially by enhanced BDNF release from oligodendrocytes and/or astrocytes.

Extending the lifetime of neural implants increases the technology reliability and reduces healthcare costs for patient populations like amputees, which may consist of 3.6 million individuals in the U.S. by 2050. Improved understanding of this technology could also suggest new therapies for TBI and neurodegenerative diseases like dementia. What is missing in the art is a system for applying ultrasound stimulation to the area surrounding an implant. While ultrasound may be known to have positive therapeutic effects, there is no system for directly applying ultrasound to an active neural implant, targeting recording sites of the implant for best results.

### SUMMARY

The present invention is directed to devices and methods for delivering acoustic stimulation to the tissue surrounding an implant with one or more electrodes that have been inserted into the tissue. The devices comprise a transducer capable of producing various frequencies of acoustic vibration and an assembly which may retain the transducer and direct the acoustic stimulation in a particular direction, namely, toward an implant. The implant electrode(s) may have one or more recording or stimulating sites thereon along the length of the electrode. The device utilizes a transducer mounted therein to produce acoustic vibrations which are delivered through a chamber having an acoustic coupling medium to target tissue. The device applies a field of acoustic vibrations to areas of tissue directly surrounding the electrode(s), at least at the recording sites thereof. In at least one embodiment, such acoustic vibrations are ultrasonic vibrations; this may also be referred to as acoustic and/or ultrasonic stimulation herein. Ultrasonic stimulation is delivered to the target tissue following insertion of the implant to reduce the body's immune system response to the implant and improve recording at the implant sensors. This response may be characterized as a foreign body response (FBR) and is a result of the insertion and presence of the electrode(s) and implant within the neural tissue.

In at least one embodiment, the implant is inserted on an oblique angle relative to the tissue surface so that the recording site(s) are directly beneath the assembly. In other embodiments, the implant is inserted substantially perpendicular to the surface of the tissue. However, in both embodiments, the implant and assembly are situated to place the target site, the tissue containing the recording sites of the electrode, within the field of a transducer capable of producing acoustic stimulation.

The assembly may consist of a series of interconnecting parts placed at the target site of the tissue. In one embodiment, the assembly consists of a base plate having a base aperture, one or more posts, a body, a chamber within the body, and a transducer housing. The assembly is defined along a longitudinal axis which is substantially perpendicular to the tissue plane. A proximal end of the assembly is located along the longitudinal axis closest to the tissue, while a distal end of the assembly is located opposite the tissue. The assembly together with the transducer define the device.

A base plate having a base aperture is positioned on or near target site tissue. The base may be mounted to the skull of a subject, which may be a human, animal, or other being, alive or dead, which may have an implant inserted therein, or directly to the subject's tissue by any mechanism providing a stable and semi-permanent attachment to the subject. The base is positioned around the implant, accommodating the implant, to target the recording sites of one or more implant electrodes. The base includes one or more posts extending parallel to the longitudinal axis of the assembly in the distal direction. The posts are secured to the base so that they may support and retain the remainder of the assembly at the target site. The posts may slidably and releasably retain the body thereon, aligning the two components with each other and with the electrodes and/or recording sites being targeted. The body includes geometrically corresponding post receivers to accept posts of the base when inserted therein. The post receivers accept the posts and align the body and base to place the chamber of the body in communication with the base plate aperture, forming a path for acoustic stimulation.

In some embodiments, a chamber is formed in the body and defined by at least one wall. The chamber retains an acoustic coupling medium, which may be polyvinyl alcohol (PVA) cryogel or other material capable of transmitting acoustic vibrations with minimal dampening or alteration to the frequency of the vibrations. The chamber wall terminates at and defines a chamber aperture toward the proximal end of the assembly and is in communication with the base aperture. The chamber is designed to direct acoustic vibration to the base aperture, and thus to a specific target site of the tissue. Being in communication with both the base aperture and transducer housing aperture, the chamber guides acoustic stimulation to the target site without obstruction.

The body further comprises contours extending parallel to the longitudinal axis toward the distal end to retain the transducer housing and align the housing with the body. An additional contour may consist of one or more alignment members extending from the body to ensure proper alignment between the transducer housing and body. The transducer housing is configured to receive and retain a transducer, such as but not limited to a piezo disc transducer or an annular or ring transducer. Specifically, an aperture formed in the housing receives at least a portion of a transducer therein. The acoustic vibrations discussed herein are produced by the transducer. Small-format, low-cost piezoelectric ceramic disc transducers with resonance near 1 MHz may be used in at least one embodiment. Transducer energy output is ideally kept below the threshold for inducing neural excitation.

In experimental therapeutic use, chronic implants may be placed within a subject from weeks to years. A critical window for treatment occurs within two weeks post-insertion. During this window, therapeutic ultrasound treatments with the above-described device are applied to the target site daily, with decreasing frequency as time progresses. For example, ultrasonic stimulation treatments are administered daily during the first week post-insertion and every other day or every three days in at least the second week post-stimulation, preferably for the remainder of the duration of implant residence in the tissue. Treatment in this critical window, also referred to as the acute or early phase, produces better long-term results in experimental subjects. These results allow for better electrode stimulation and better recording of brain activity at the recording sites of the electrode, as shown in FIGS. 18A-18B and as described in more detail below.

To use the device, first an implant is inserted into a subject. This implant may be inserted at an oblique angle as described above. The transducer may have been attached to the housing at any point during the above-described assembly process. Once assembled, the transducer may be selectively activated for limited periods of time to avoid heating the tissue via excess acoustic stimulation. In one exemplary embodiment, the transducer may be activated for periods of 5 minutes, with 5-minute rest periods between activations. This may continue for a period of 15 minutes to complete a treatment cycle, and may be repeated on subsequent days according to the above protocol. The recording sites of the implant are targeted during activation, ideally being at a focal point of the acoustic field. During activation, the recording sites may cease collecting data, as the acoustic stimulation may introduce artifacts into data output.

The ultrasonic field produced by the transducer may be altered by a variety of factors, including but not limited to the geometry of the transducer, frequency of vibration, thickness of the transducer, acoustic lens application focusing the stimulation, concentric annular piezoelectric elements being selectively excited, and by other factors known in the art.

Some embodiments may utilize an annular, or ring-shaped, transducer. The annular transducer, in combination with a correspondingly shaped assembly, allows the body and transducer housing, to define a passage therethrough which allows an implant to be inserted into a subject substantially perpendicular to the tissue. The operation of the device is substantially similar to the operation of the disc-shaped transducer embodiment described further herein. The chamber encircles the passage, forming an annular chamber which may be substantially cylindrical in form, without angling the acoustic field in any particular direction to maximize the overlap between acoustic fields from opposing sides of the annular transducer.

The device, together with its particular features and advantages, will become more apparent from the following detailed description and with reference to the appended drawings.

### DESCRIPTION OF THE DRAWINGS

FIG. 1 is a perspective view of a first illustrative embodiment of the device of the present invention, having a disc transducer, placed in proximity to neural tissue and showing the placement of an implant electrode relative thereto.

FIG. 2 is an exploded top perspective view of the device of FIG. 1.

FIG. 3 is an exploded bottom perspective view of the device of FIG. 1. FIG. 4A is a top perspective view of the assembled device of FIG. 1 shown mounted on a stereotaxic frame.

FIG. 4B is a detail view of the assembled device of FIG. 4A.

FIG. 5 is a side cross-sectional view of the device of FIG. 1, showing the chamber and interactions of the posts with the body.

FIG. 6 is a top view of the device of FIG. 1, shown without a transducer, exposing the chamber.

FIG. 7 is an illustrative diagram of an ultrasonic field produced by the device of FIG. 1 with reference to an inserted implant electrode.

FIG. 8 is a diagram of the ultrasonic field shown in FIG. 7 with an adjustment to the field by changing one or more parameters of the transducer.

FIG. 9 is a perspective view of a second illustrative embodiment of the device of the present invention, having an annular transducer, in proximity to neural tissue and showing the placement of an implant electrode relative thereto.

FIG. 10 is an exploded top perspective view of the device of FIG. 9.

FIG. 11 is an exploded bottom perspective view of the device of FIG. 9.

FIG. 12 is a top perspective view of the assembled device of FIG. 9 shown mounted on a stereotaxic frame.

FIG. 13 is a side cross-sectional view of the device of FIG. 9, showing the chamber and interactions of the posts with the body.

FIG. 14 is a top view of the device of FIG. 9, shown without an annular transducer, exposing the chamber.

FIG. 15 is an illustrative diagram of an ultrasonic field produced by an annular transducer of FIG. 9 with reference to an inserted implant electrode.

FIG. 16 is an illustrative diagram of an ultrasonic field produced by an angled annular transducer with reference to an inserted implant electrode.

FIG. 17 is an illustrative diagram of an ultrasonic field produced by concentric annular transducers with reference to an inserted implant electrode.

FIG. 18A are graphical data of implanted electrodes treated with the device and method of the present invention as described in the Example, showing more active recording channels from the treatment compared to controls.

FIG. 18B are graphical data of implanted electrodes treated with the device and method of the present invention as described in the Example, showing increased signal-to-noise ratio from the treatment compared to controls.

Like reference numerals refer to like parts throughout the several views of the drawings.

### DETAILED DESCRIPTION

As shown in the accompanying drawings, the present invention is directed to a device 100 for delivering acoustic stimulation to an implant 10, having one or more electrodes 12, that has been inserted into tissue 5. The device 100 comprises a transducer 142 capable of producing various frequencies of acoustic vibration and an assembly 102 which may retain the transducer 142 and direct the acoustic stimulation in a particular direction, namely, toward an implant 10, and more specifically to the electrode(s) 12 thereof and at least one recording site 14. The implant 10 electrode(s) 12 may have one or more recording sites 14 thereon along the length of the electrode 12. The device 100 applies a field 160 of acoustic vibrations to areas of tissue 5 in contact with electrode(s) 12, at least at the recording sites 14 thereof, which is referred to herein as the target site 7. In at least one embodiment, such acoustic vibrations are ultrasonic vibrations; this may also be referred to as acoustic and/or ultrasonic stimulation herein. Though described in terms of neural tissue herein for the sake of simplicity, the tissue 5 may be any type of tissue, such as, but not limited to, neural tissue, connective tissue, epithelial tissue, and muscle tissue. In at least one embodiment, the tissue 5 is neural tissue, including but not limited to brain tissue (including cortical and/or deep brain structures), the spinal cord, and peripheral nerves. Tissue 5 may be that of any animal having neural tissue 5, such as but not limited to humans, non-human primates, rodents, rabbits, and other animals used in animal modeling. The device 100 may be mounted directly onto a subject, positioned to capture the recording sites 14 of the implant 10 within its field 160 of ultrasonic stimulation.

Ultrasonic stimulation is delivered to the target tissue 5 following insertion of the implant 10 to reduce the body's response to the implant 10 and improve recording at the implant sensors 14. This response may be characterized as a foreign body response (FBR) and is a result of the insertion and presence of the electrode(s) 12 and implant 10 within the neural tissue 5. FBR is an inflammatory response causing neural tissue 5 damage and glial scarring, reducing the effectiveness of the implant sensors 12. The device 100 of the present invention utilizes a transducer 142 mounted therein to produce ultrasonic stimulation which is delivered to target tissue 7 through a chamber 122 having an acoustic coupling medium 126. Without wishing to be bound by any theory, it is believed that the application of low-power therapeutic ultrasound may induce the release of endogenous brain derived neurotrophic factor (BDNF) from within neural tissue 5. BDNF, an anti-inflammatory neuroprotective factor, along with other neurotrophins, may limit the inflammatory FBR response caused by implant 10 insertion at least in part by blocking caspase, an enzyme involved in cell death.

The device 100 consists of an assembly 102 placed on and/or secured to the body of a subject in proximity to a target site 7 for the acoustic stimulation. This target site 7 is the area of tissue 5 having an implant 10 inserted therein. The implant 10 may consist of one or more electrodes 12 having elongate length and at least one recording site 14 thereon. Specifically, the target site 7 is the electrode 12 and at least one recording site 14 thereof, which may be located anywhere along the length of the electrode 12. In one embodiment, a recording site 14 may be located at a distal tip of the electrode 12. In another embodiment, recording sites 14 may be spaced apart from one another along the length of the electrode 12. These recording sites 14 may measure different aspects of electrical impulses transmitted by the electrodes 12 to the adjacent neural tissue 5 and may collect various data associated with brain activity and such impulses. For instance, in at least one embodiment, the recording site(s) 14 may measure electrical potentials encoding components of neural activity spanning a broad frequency range, including frequencies up to 5 kHz. These electrical potentials may range from low-frequency, large-amplitude, spatially propagating electrical potentials, to local field potentials (LFPs) associated with arousal and behavior, to spatially discrete, high-frequency, single and multi-unit action potentials generated by individual neurons located close to the electrode recording site. Electrical potentials can be recorded simultaneously as a single broadband signal and then components may be individually isolated using common bandpass filtering and feature detection algorithms, creating high dimensional datasets.

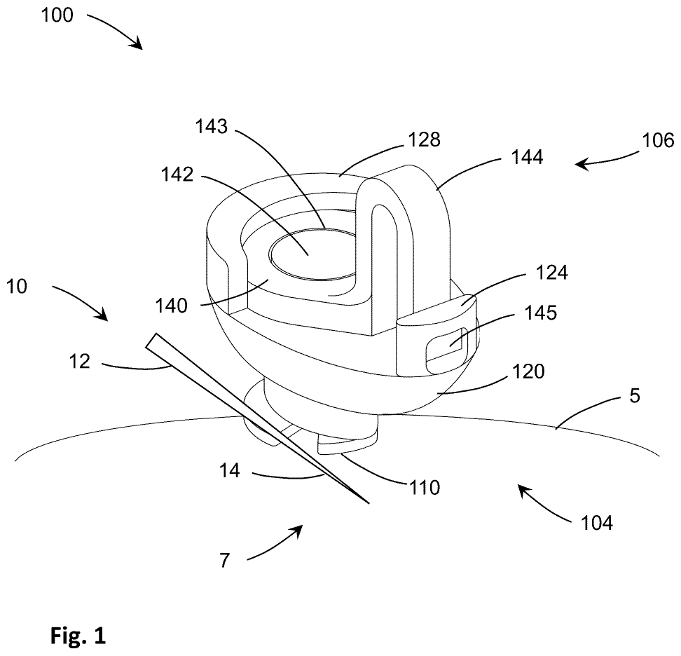

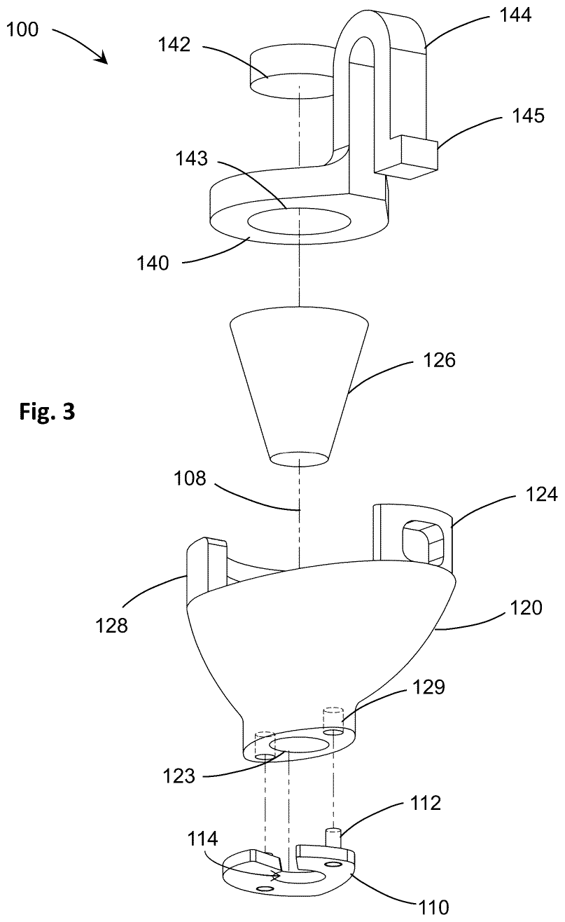

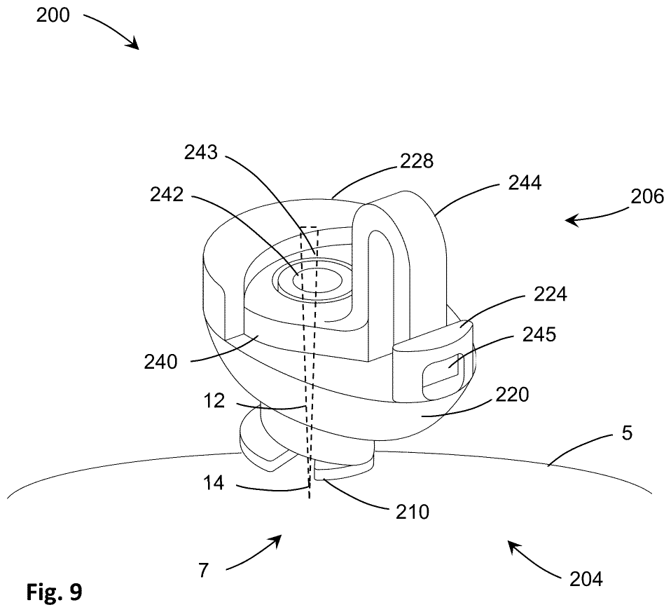

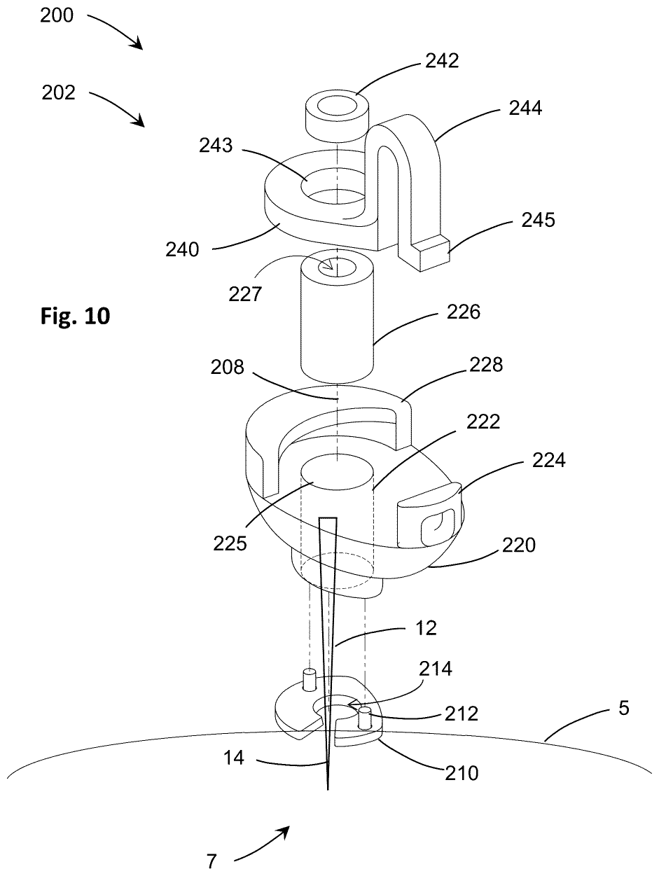

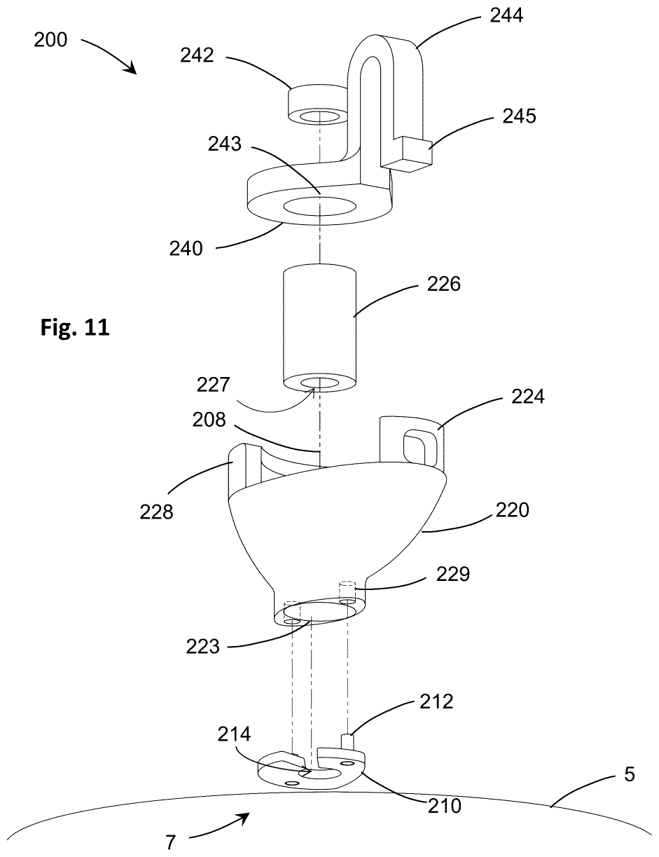

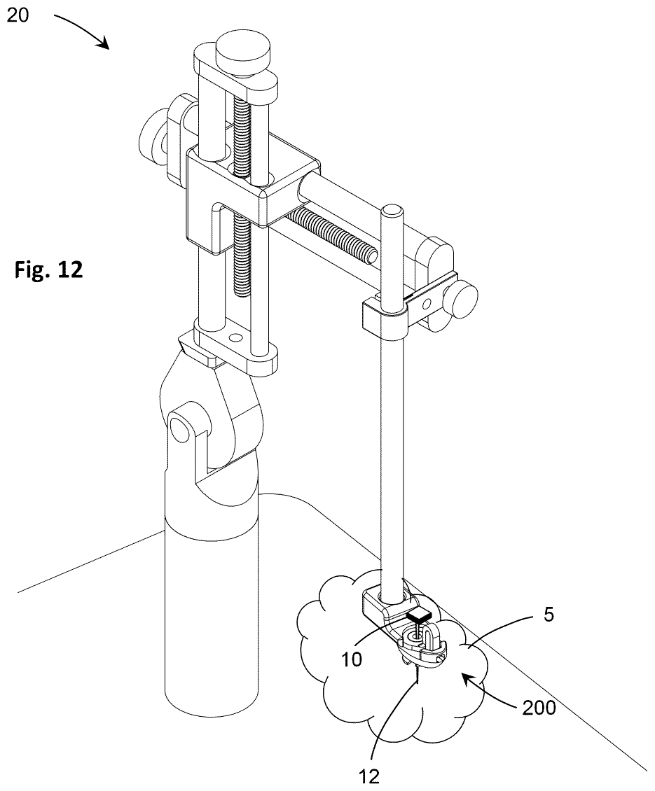

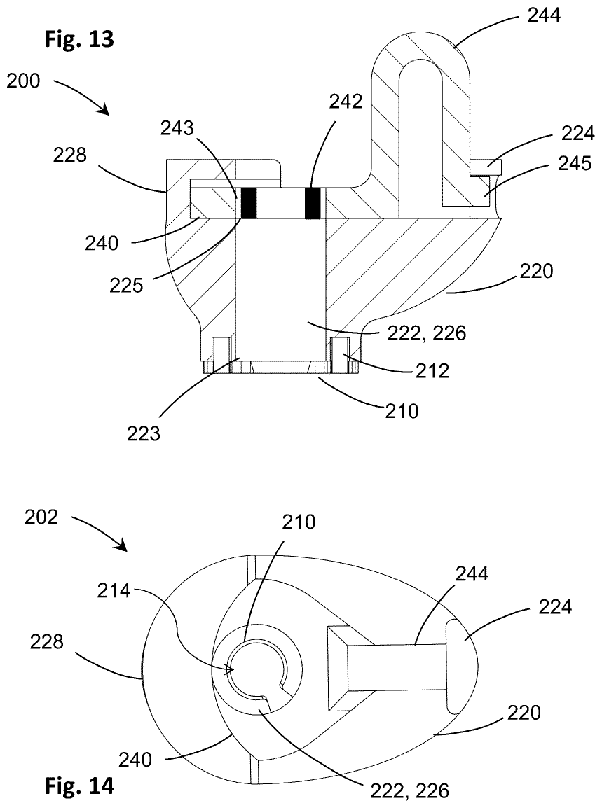

In at least one embodiment, such as shown in FIGS. 1-8, the implant 10 is inserted on an angle so that the recording site 14 sits directly beneath the assembly 102. The implant 10 may be inserted at any oblique angle relative to the surface of the tissue 5, such as but not limited to 5, 10, 20, 30, 40, 45, 50, 60, 70, 80, and 85 degrees. In other embodiments, such as the embodiment shown in FIGS. 9-17, the implant 10 is inserted substantially perpendicular to the surface of the tissue 5. However, in both embodiments, the implant 10 and assembly 102 are situated to place the target site 7, the tissue 5 containing the recording sites 14 of the electrode 12, within the field 160 of a transducer 142 capable of producing acoustic stimulation. Implant 10 electrodes 12 may be placed at any depth relative to the surface of the tissue 5.

Without limitation, a subset of neural implants 10, penetrating intracortical microelectrode arrays 12, are composed of multiple penetrating members with typical cross-sectional diameters in the range of 25-100 μm and are typically implanted 0.25-2 mm into brain tissue 5, but sometimes as deep as several centimeters when targeting deep brain structures in some subjects. The recording sites 14 are relatively small with high impedance (>100kΩ, a requirement for recording unit activity from individual neurons. Variations in penetrating electrode technologies include insulated metallic microwires, micromachined high density 3-D electrode arrays such as the Utah electrode array (Blackrock Microsystem, Salt Lake City, UT) that are similar in geometry to microwire electrode arrays, and planar thin-film microelectrode arrays like Michigan probes, produced by NeuroNexus Technologies (Ann Arbor, MI), composed of silicon or polymer substrates with multiple electrode sites along the penetrating members. However, the consistency in performance of penetrating neural microelectrode arrays is highly variable. For instance, a group at University of Michigan now has a team of individuals experienced in implanting their microelectrode arrays in subjects, and approximately 67% of the time the implants record unit activity for 3-6 months or more. However, the remaining 33% of the electrode arrays often fail at around 6 weeks, suggesting that if the microelectrode arrays can make it beyond this critical window, they could record neural activity indefinitely. The present device 100 may be used with any of these types of implants 10.

The assembly 102 may consist of a series of interconnecting parts placed at the target site 7 of the tissue 5. In the embodiment shown at FIGS. 1-8, the assembly 102 consists of a base plate 110 having a base aperture 114, one or more posts 112, a body 120, a chamber 122 within the body 120, and a transducer housing 140. The assembly 102 is defined along a longitudinal axis 108 which is substantially perpendicular to the tissue 5 surface. A proximal end 104 of the assembly 102 is located along the longitudinal axis 108 closest to the tissue 5, while a distal end 106 of the assembly 102 is located opposite the tissue, as shown in FIG. 1. The assembly 102 together with the transducer 142 defines the device 100.

### Disc Embodiment

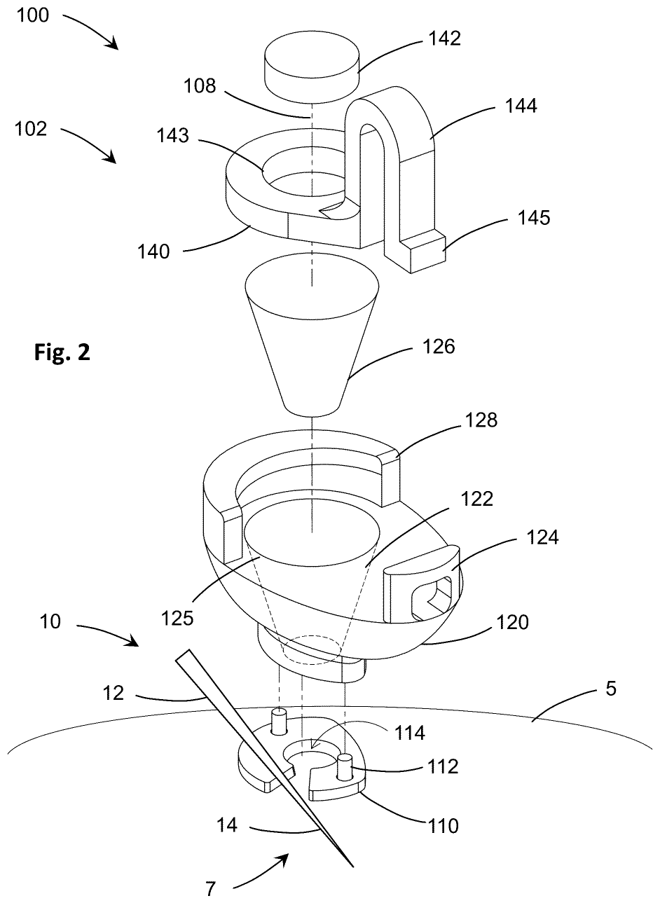

In a first embodiment shown in FIGS. 1-8, and particularly as shown in FIG. 1, the device 100 includes a base plate 110 having a base aperture 114 that is positioned on or near target site 7 tissue 5. The base plate 110 may consist of a plate or any other substantially planar surface and can have any shape suitable for supporting the remainder of the assembly 102. The terms “base” and “base plate” may be used interchangeably herein. The base 110 may be mounted to the skull of the subject or directly to the subject's tissue 5 by any mechanism providing a stable and semi-permanent attachment to the subject, such as but not limited to anchoring by dental acrylic or a similar anchoring substance, by screw attachment, by a combination of dental acrylic and screw attachment, or by any similar mechanism. The base 110 is mounted to the subject at a point where neural tissue 5 is at least partially exposed, having some layers of skin, bone, or other tissue removed to expose the target site 7. In one exemplary embodiment, the base 110 is mounted to the subject via dental acrylic. The base 110 may at least partially encircle the target site 7, at least on the surface above the target site 7. As shown in FIG. 2, the base 110 may have a substantially annular shape defining a base aperture 114 therein. However, in some embodiments, the base 110 does not completely encircle the target site 7, leaving an opening in its substantially annular form to allow access to the site 7 by an implant 10 which may be inserted into the tissue 5 at an oblique angle. The base 110 is positioned around the implant 10, accommodating the implant 10, to target the recording sites 14 of one or more implant electrodes 12.

As shown in FIGS. 1-2, the base 110 includes one or more posts 112 extending outwardly parallel to the longitudinal axis 108 of the assembly 102 toward the distal end 106. In at least one embodiment, the posts 112 are secured to the base 110, though in other embodiments the posts 112 may be integrally formed with the base 110. Together with the base 100, the posts 112 support and retain the remainder of the assembly 102 and properly position the device 100 at the target site 7. Posts 112 may be made of any suitable material for retaining the assembly 102 on the subject but need not be the same material as the remainder of the assembly 102. As shown in FIG. 1, posts 112 may be located on the base 110 on either side of the base aperture 114, positioning the acoustic chamber 122 of the assembly 102 in communication with the target site 7 tissue 5. The posts 112 may slidably and releasably retain the body 120 thereon, aligning the base 110 and body 120 with each other and with the recording sites 14 being targeted. As shown in the embodiment of FIG. 2, the posts 112 may be cylindrical in nature, but in other embodiments may be a projection or contour extending from the base 110 in any geometric shape that is able to align and retain the body 120 thereon.

In some embodiments, as shown in FIGS. 1-6, the body 120 is received on and supported by the base 110, aligned properly by the posts 112. The body 120 includes post receivers 129 geometrically corresponding to accept posts 112 of the base 110 when inserted therein. In certain embodiments, the post receivers 129 are matingly configured to the posts 112 and conform to the dimensions thereof. The post receivers 129 accept the posts 112 and align the body 120 and base 110 together to position the chamber 122 of the body 120 in communication with the base plate aperture 114, forming a path for acoustic stimulation transmission, as shown in FIG. 6.

The body 120 includes a chamber 122 formed in the body 120 which is defined by at least one wall 121. The chamber 122 may be cylindrical, conical, or any other shape suitable for holding and retaining material therein and/or directing acoustic stimulation therethrough. The chamber 122 receives and retains an acoustic coupling medium 126 therein, which may be polyvinyl alcohol (PVA) cryogel or other material capable of transmitting acoustic vibrations with minimal dampening or alteration to the frequency of the vibrations. The chamber 122 is capable of retaining acoustic coupling medium 126 in liquid, solid, or semi-solid form such as gels like PVA cryogel. Solid and semi-solid acoustic coupling medium 126 may be formed to conform to the dimensions and shape of the chamber 122, by means suitable for the medium, such as but not limited to by molding, extrusion, 3D printing, milling, and various other techniques. PVA cryogel has mechanical and coupling properties that provide good acoustic coupling for transmission of therapeutic ultrasound. In at least one embodiment, the acoustic coupling medium 126 may be 3D printed conical PVA hydrogel being 10% weight by volume PVA made using two freeze-thaw cycles and having a molecular weight of 78,000 (Polysciences, Inc., Warrington, PA), though other PVA compositions with different weight by volume and molecular weights are also contemplated herein. In some embodiments, the acoustic coupling medium 126 does not fill the chamber 122 but rather lines the chamber. In at least one embodiment, however, an acoustic coupling medium 126 may fill the chamber 122 to transmit acoustic stimulation therethrough. Preferred cone geometry consists of a 3 mm diameter flat cone tip, an 8 mm base, and 10 mm height. However, the cone may have any geometry sufficient to accommodate use of a desired transducer 142. Indeed, in certain embodiments the acoustic coupling medium 126 may be cylindrical in shape, having an outer diameter similar to the inner diameter of the chamber 122.

The chamber wall 121 terminates at and defines a chamber aperture 123 toward the proximal end 104 of the assembly 102 and is in communication with the base aperture 114. In certain embodiments, the chamber aperture 123 and base aperture 114 may have similar or substantially the same diameters. This allows the acoustic coupling medium 126 retained within chamber to contact tissue 5 through the base 110. The coupling medium 126 may be fitted to the chamber 122, extending between the chamber aperture 123 and a chamber opening 125 defined by the body 120 at its distal end 106. The chamber wall 121 terminates at the chamber opening 125. In some embodiments, the chamber opening 125 and chamber aperture 123 may have similar or substantially the same diameters. In at least one embodiment, as shown in FIGS. 1-8, the chamber aperture 123 may have a smaller diameter than the chamber opening 125. In certain embodiments and as shown in FIGS. 2 and 6, the chamber opening 125 is aligned with the transducer housing 140 when the body 120 and housing 140 are assembled. The chamber 122 may be formed from the coupling medium 126, or the medium 126 may be poured into or otherwise placed within the chamber 122. The chamber 122 is designed to direct acoustic vibration to a specific target site 7 of the tissue 5. Being in communication with both the base aperture 114 and transducer housing aperture 143, the chamber 122 and acoustic coupling medium 126 therein guides acoustic stimulation to the target site 7 without obstruction, as shown in FIG. 6.

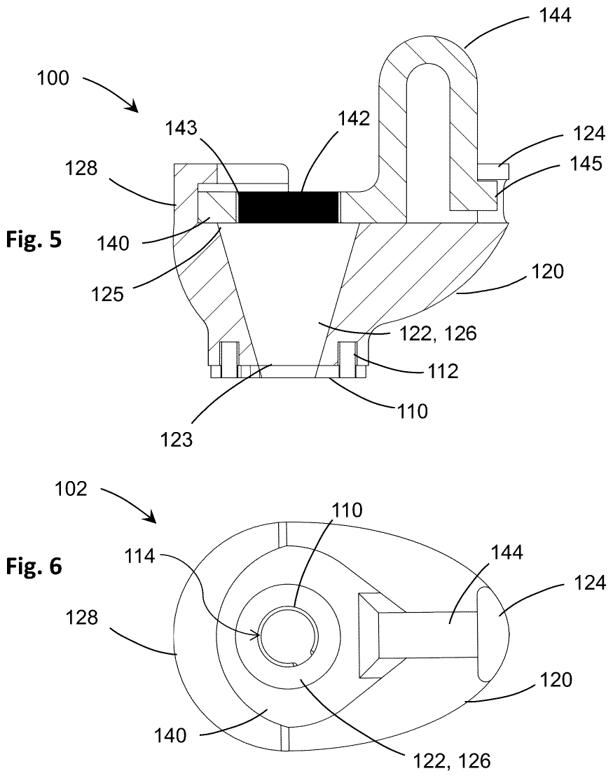

The body 120 further comprises contours extending parallel to the longitudinal axis 108 toward the distal end 106 to align and secure the transducer housing 140 to the body 120. For instance, in some embodiments as shown in FIGS. 2 and 5, one contour may be a retention clip receiver 124 extending from the body to receive a portion of the housing 140 therein, such as but not limited to the retention clip 144 having an insert 145 extending therefrom. This retention clip receiver 124 may be formed on the body 120 at any location and may optionally be formed adjacent to the chamber opening 125. The retention clip receiver 124, as shown, defines an aperture through which the retention clip insert 145 may be releasably received, aligning the housing 140 with the body 120. However, the retention clip receiver 124 may consist of any suitable contour or configuration to receive and selectively restrain a portion of the housing 140 retention clip 144 therein. Additional contours may include one or more alignment members 128 extending from the body 120 to ensure proper alignment between the transducer housing 140 and body 120. This alignment member 128 may be formed adjacent to the chamber opening 125 and may be curved, positioned or otherwise configured similarly to at least a portion of the transducer housing 140. As shown in the embodiment of FIG. 2, this alignment member 128 geometrically corresponds to the housing 140 and forms a backstop to align the transducer 140 with the chamber opening 125, and thus with the acoustic coupling medium 126 therein, and to maintain tension between the retention clip 144 and receiver 124 to keep the housing 140 secured to the body 120.

As shown in FIGS. 1-6, the transducer housing 140 is configured to receive and retain a transducer 142, such as but not limited to a disc transducer 142. Specifically, a housing aperture 143 formed in the housing 140 receives at least a portion of a transducer 142 therein. In some embodiments, the transducer 142 may be retained by the housing 140 via frictional fit. In other embodiments, the transducer 142 may be retained by a lip extending from the housing 140, by glue or other adhesive, by screw, clamp or other means sufficient to retain the transducer 142 in the housing 140 during use. The present assembly 102 is dynamic and able to receive transducers 142, 242 with different dimensions and geometry either through a single universal transducer housing 140 or a multitude of transducer housings 140 each adapted to receive a set of transducers 142 having a particular geometry. The housing aperture 143 of the housing 140 aligns with the chamber opening 125 of the chamber 122 within the body 120 such that the portion of the transducer 142 retained in the housing aperture 143 is in communication with and contacting the acoustic coupling medium 126 within the chamber 122. Acoustic vibrations generated by the transducer 140, therefore, may be transmitted to the acoustic coupling medium 126.

In the certain embodiments described above, the housing 140 is selectively attachable to the body 120 by contours on the surface of the body 120 that may correspond to the geometry of the housing, such as but not limited to a retention clip receiver 124 and alignment member 128. As shown, the housing 140 includes a retention clip 144, and retention clip insert 145 extending therefrom, which is configured to align the housing aperture 143, and therefore the transducer 142, with the chamber 123 below. The retention clip 144 is selectively deformable so the housing 140 to be removable from the body 120 when desired. The assembly 102 formed by the base 110, body 120, and housing 140 may be selectively disassembled as needed through the various attachment mechanisms discusses herein, as well as by frictional fit, clips, corresponding contours, or other similar mechanisms. At least a portion of the retention clip 144, such as the arm 146, may be formed of resilient material capable of deforming temporarily to facilitate movement of the clip insert 145 into and out of the retention clip receiver 124. Examples include, but are not limited to, plastics, thermoplastics and polymers of various types.

The device 100 also includes a transducer 142 capable of generating acoustic vibrations when activated. The terms “transducer,” piezoelectric element,” and “piezo” may be used interchangeably herein to refer to a device generating acoustic vibrations when activated. As shown in FIG. 6, the base plate aperture 114 which frames the target site 7 of acoustic stimulation, is in communication with the transducer 140 via the chamber 122, which directs such stimulation to the target site 7. In at least one embodiment, as shown in FIGS. 1, 4, and 5, small-format, low-cost piezoelectric ceramic disc transducers 142 with resonance near 1 MHz may be used (APC International, Ltd, Mackeyville, PA). In other embodiments, as described below, piezoelectric elements having various geometries may be used, such as but not limited to annular and angled piezoelectric elements 242. Transducers 142, 242 used in the device 100 described herein may preferably produce acoustic vibrations of frequencies between 200 kHz and 5 MHz, preferably 500 kHZ-3 MHz, more preferably 1.0-2.2 MHz, and, in one exemplary embodiment, 1.13 MHz. However, transducers 142, 242 may be used with a range of potential frequencies including up to 2 MHz, up to 5 MHz or values in the tens of megahertz, specifically between 5 and 20 MHz. Regarding transducer 142, 242, the spatial peak temporal average intensity (I_{SPTA}) is preferably equal to 0.5 W/cm². I_{SPTA} being the maximum intensity averaged over the pulse repetition period within the acoustic field 160, indicating the thermal effect of ultrasonic stimulation on tissue 5 (i.e., the amount of heat delivered to target tissue 7 by a transducer 142, 242). The threshold I_{SPTA} value of 0.5 W/cm² has been found to induce BDNF release without crossing neural activation thresholds. Transducer 142, 242 output, ideally may be below a threshold to elicit a brain response to the stimulation, avoiding creating a twitch in the subject. However, other I_{SPTA} value thresholds are contemplated herein, such as but not limited to values between 0.01-2.5 W/cm², preferably 0.1-2 W/cm², and, in one exemplary embodiment, 0.5 W/cm². Transducer 142, 242 voltage may be between 100 and 600 V, preferably above 200 V, or, in one exemplary embodiment, 280 V. Duty cycle percentage between 0.5% and 20%, but preferably near 5%, and, in one exemplary embodiment, 4.2%

Transducers 142, 242 as described herein may have various geometries which may affect the acoustic field 160 produced by each transducer 142, 242, and therefore vary the stimulation of target tissue 7 with variation of the transducer 142, 242. Transducer 142, 242 diameter may measure between 2-14.5 mm, preferably between 4.9-8 mm, or more preferably 6.4 mm. Pulses generated by the transducer 142, 242 may have durations between 5-200 ms, preferably having 22 ms durations. Transducer 142, 242 thickness may fall between 0.2 and 6 mm, preferably 1-2.2 mm. During treatment, the transducer 142, 242 may reach a maximum temperature of 27.6° C., but may ideally run at temperatures below 38.5° C., preferably below 38° C., to avoid tissue damage.

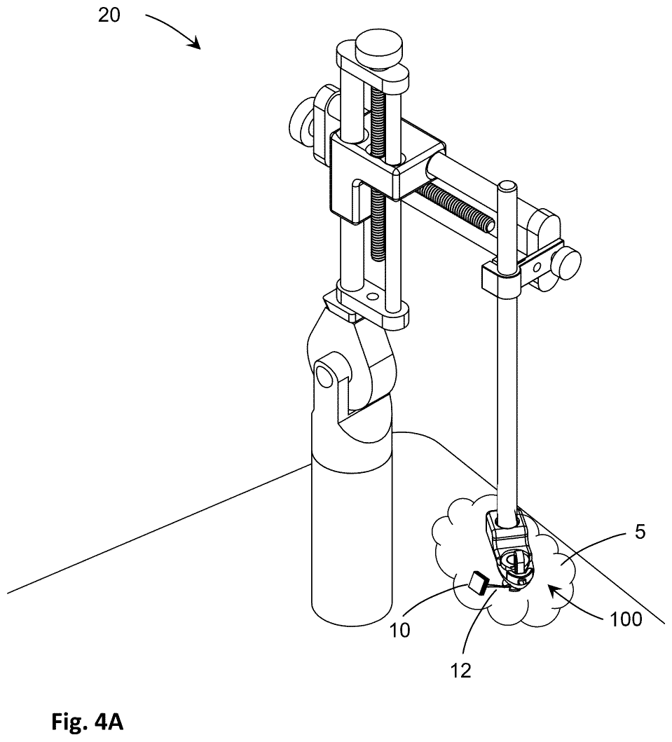

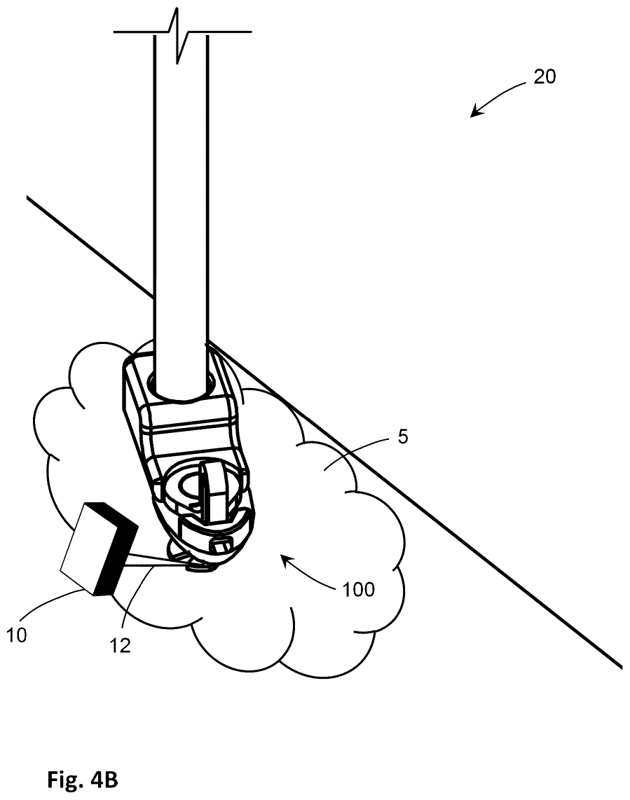

In some embodiments, the device 100, 200 may be mounted to a stereotaxic frame 20 when in use, as shown illustratively in FIGS. 4A-B and 12. A frame 20 may hold the device 100, 200 in proximity to the subject, or the device may be independently mounted to the subject. In some embodiments, the frame 20 may attach to the device 100, 200 via an adapter configured to hold the device by either wrapping around the device 100, 200 or by being inserted between the pieces of the assembly 102, 202 itself, such as by attaching to one or more posts 112, 212 of the assembly 102, 202. In alternate embodiments, the frame 20 may attach by screw, clamp, adhesive, press-fit, or any other similar method. As shown in FIGS. 4A-B, the device 100 may be used in combination with an implant 10 having at least one electrode 12 or an array of electrodes 12. In experimental therapeutic use, implants 10 may be placed within a subject for up to six weeks. A critical window for treatment occurs within two weeks post-insertion, also referred to as the acute or early phase of implant residence. During the first week of this window, therapeutic ultrasound should be applied to the target site 7 daily, with decreasing frequency as time progresses. For example, in a second week ultrasonic stimulation may be administered every other day or every three days.

To use the device 100, first an implant 10 is inserted into a subject. In at least one embodiment, this implant 10 is inserted into tissue 5 at an oblique angle as described above. Importantly, the oblique angle of the implant 10 relative to the assembly 102 allows the transducer 140 to target the recording sites 14 when positioned on the tissue 5, placing the sites 14 within the ultrasonic field 160 generated by the device 100. The location of recording sites 14 along an electrode 12, depth of insertion of an implant 10 and the angle of insertion of the implant 10, allow a user to mathematically determine the target site 7 for ultrasonic stimulation and accordingly attach the base plate 110 to the subject with the base plate aperture 114 aligned with the specific target site 7. In one embodiment, the base plate may be attached directly to the skull of the subject, or may be indirectly mounted to the subject adjacent to the target site 7, as described in further detail above. The base 110 and posts 112 receive the body 120 thereon, slidably retaining the body 120 in alignment with the base aperture 114 so that the chamber 122 and aperture 114 are in communication with one another. The body 120 may or may not be attached to the transducer housing 140 prior to attaching to the base 110. The transducer housing 140 is connected to the body 120, aligning the housing aperture 143 with the chamber 122. The retention clip 144 may be temporarily reversibly deformed by a user to allow the clip insert 145 to slide into the retention clip receiver 124, releasing the clip 144 when the insert 145 and receiver 124 are aligned. The insert 145 and receiver 124 holding the body 120 and housing 140 statically together, aided by the additional contours 128 of the body. The transducer housing 140 may or may not contain the transducer 142 therein prior to attachment to the body 120. In any case, the device 100 may be entirely or partially assembled with the base plate 110 prior to attachment to a subject.

In some embodiments, the transducer 142 may have been attached to the housing 140 at any point during the above-described assembly process. In at least one embodiment, once assembled, the transducer 142 may be selectively activated for limited periods of time to avoid heating the tissue 5 via excess acoustic stimulation. For instance, in one exemplary embodiment, the transducer 142 may be activated for periods of 5 minutes, with 5-minute rest periods between activations. This may continue for a period of 15 minutes to complete a treatment cycle. Other embodiments contemplate different periods of activation and rest, and different overall treatment cycle times, which may be greater or less than those disclosed above. Without limitation, a treatment cycle may have periods of activation for a time in the range about 1 to 15 minutes and periods of rest for a time in the range about 1 to 15 minutes, repeating the periods of activation and rest between 2 to 10 times The recording sites 14 of the implant 10 are targeted during activation, ideally being at a focal point of the acoustic field 160. During activation, the recording sites 14 may cease collecting data, as the acoustic stimulation may introduce artifacts into data output.

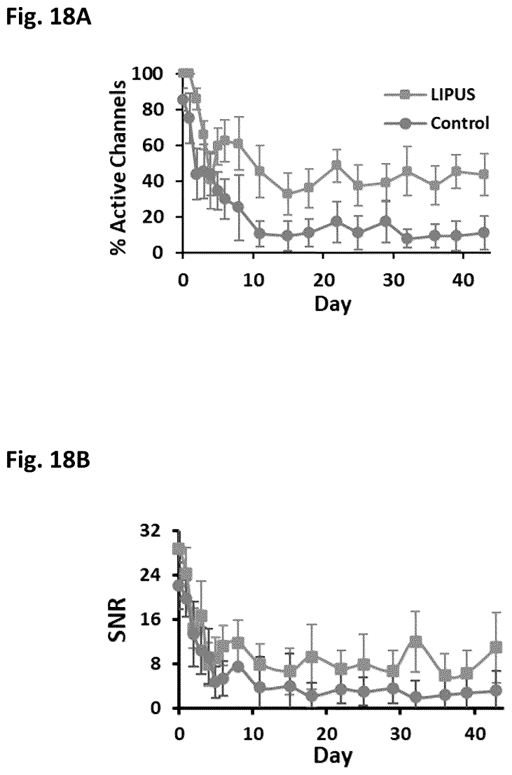

In at least one embodiment, the device 100 may be used to reduce foreign body response in the subject through the following steps. First, the method begins by positioning the device 100 in contact with the tissue 5 and in proximity to the target site 7. Then, the method includes generating acoustic vibrations by activating the transducer 142, 242 for a predetermined period of time, transmitting said acoustic vibrations to the target site 7. Sufficient acoustic vibrations may be applied to the target site 7 to reduce immune system foreign body response in the subject where the electrode 12 contacts the target tissue 7. This may be demonstrated by more active recording channels and/or better signal to noise measurements from recording sites for the duration of the implantation following treatment, such as shown in FIGS. 18A-18B and described in the Example below. The vibrations may be of a frequency and intensity sufficient to stimulate release of at least one endogenous neurotrophic factor in the target tissue 7. In some embodiments, these acoustic vibrations are in the ultrasonic frequency range. Acoustic vibrations may be pulsed, having a duration in the range of about 5 to 200 milliseconds.

In some embodiments, treatment may consist of activating said transducer 142 for a predetermined period of time, turning the transducer 142 on for 5 minutes, then off for 5 minutes, then on for 5 minutes for a total treatment time of 15 minutes. The above steps may be repeated once daily for the first week following implantation of the electrode 12 and once every two or three days during the second week following implantation of the electrode 12. Acoustic vibrations generated during treatment create an acoustic field 160 of said acoustic vibrations at the target site 7, the acoustic field 160 surrounding at least a portion of the electrode implanted in the target tissue 7. This field 160 acoustic field comprises a near field 162 and a far field 164 separated by a transition point 166, where the far field 164 may have a wider diameter than the near field 162. In some embodiments, the field 160 may be modulated by changing the frequency of said acoustic vibrations and the diameter of the transducer 142. However, the field 160 may be modulated by altering any one or more of the above-described operative parameters, such as but not limited to frequency, voltage, temperature, transducer geometry, duty cycle, pulse duration, or I_{SPTA}.

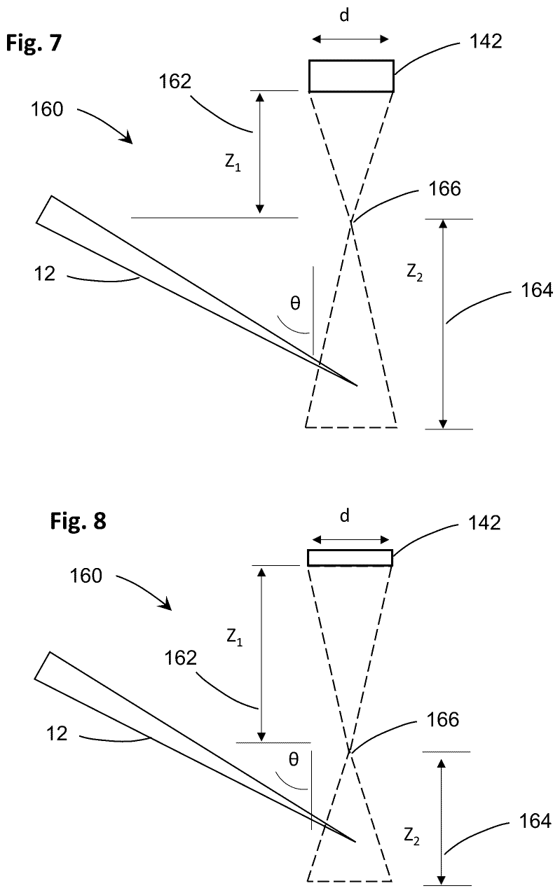

As shown in FIGS. 7-8, the ultrasonic field 160 produced by the transducer may be altered by a variety of factors, including but not limited to the geometry of the transducer, frequency of vibration, thickness of the transducer, acoustic lens application focusing the stimulation, concentric annular piezoelectric elements being selectively excited, and by other factors known in the art. The acoustic field 160 is defined by a near, or proximal, field 162 located adjacent to the transducer 140 and a far, or distal, field 164 located past a transition point 166, penetrating deeper into target tissue 7. The field 160 is approximately the diameter of the transducer 142 within the near field 162 and diverges past the transition point 166 to have increasingly greater diameter than the transducer 140. This divergence from the transition point 166 in the far field 164 is defined by a divergence angle, shown as θ in FIGS. 7-8. Increased diameter of the transducer 140 correspondingly increases the diameter of the near and far fields 162, 166. Resonance frequency of the transducer 142 varies as the transducer 142 thickness varies, where the piezoelectric element 142 operates as a half-wavelength resonator, the frequency of ultrasound produced may be defined by the equation:

$$f = \frac{v}{2 t} \qquad ( 1 )$$

where v is the sound velocity in the piezoelectric element 142 material (often being near 4,000 m/s), and t is the thickness of the piezoelectric element 142. Therefore, a thicker material produces a lower frequency.

### Annular Embodiment

In a second embodiment, the device 200 as shown in FIGS. 9-17, may utilize an annular, or ring-shaped, transducer 242. The annular transducer 242, in combination with a correspondingly shaped assembly 202 shown in FIGS. 9-14, allows the body 220 and transducer housing 240, to define a passage 227 therethrough which allows an implant 10 to be inserted into a subject substantially perpendicular to the tissue 5. The operation of the device 200 is substantially similar to the operation of the disc-shaped transducer 142 embodiment of the device 100 described in detail above.

In certain embodiments, the base plate 210 may be mounted to the subject in substantially the same manner as described above with reference to the first embodiment, accommodating the implant 10 through a base plate aperture 214 therein. Posts 212 extending from the base 210 may be configured to fit within post receivers 229 defined in the body 220 and to receive the body 220 thereon. The body 220 may have a substantially similar configuration to the body 120 described in more detail above, with the exception of a chamber 222 conforming to the contours of the annular transducer 242. In certain embodiments, the chamber 222 containing an annular acoustic coupling medium 226 encircles the passage 227, forming an annular chamber 222 which may be substantially tubular in form, without angling the acoustic field 260 in any particular direction to maximize the overlap between acoustic fields 260 from opposing sides of the annular transducer 242. In some embodiments, the chamber 222 may also be angled, similar to the chamber 122 shown in FIG. 5. The chamber having an annular aperture 223 at the proximal end 204 of the assembly 202, adjacent to the tissue 5, in fluid communication with the base aperture 214. Opposite the aperture 223, a chamber opening 225 at the distal end 206 of the body 220 substantially conforms to the geometry of an annular transducer 242 and is in communication with the transducer 242 when the device 200 is in use. The body 220 has a retention clip receiver 224 and alignment member 228 extending therefrom to receive and restrain the transducer housing 240 and the retention clip 244 insert 245.

A transducer housing 240 in substantially the same form as the disc transducer housing 140, described in more detail above, receives an annular transducer therein 242 and attaches in alignment with the chamber 222 below. A user may reversibly deform the retention clip 244 and place the housing 240 on the base between the clip receiver 224 and alignment member 228.

To use the device 200, first an implant 10 is inserted into a subject. This implant 10 is inserted substantially perpendicularly to the tissue 5 surface. The passage 227 defined by the assembly 202 allows the transducer 240 to target the recording sites 14, placing the sites 14 within the ultrasonic field 260. The location of recording sites 14 along an electrode 12, and depth of insertion of an implant 10 allow a user to mathematically determine the target site 7 for ultrasonic stimulation and accordingly attach the base plate 210 to the subject. The base plate aperture 214 being aligned with the specific target site 7. In one embodiment, the base plate 210 may be attached directly to the skull of the subject, or may be indirectly mounted to the subject adjacent to the target site 7, as described in further detail above. The base 210 having posts 212 receives the body 220 thereon, slidably retaining the body 220 in alignment with the base aperture 214 so that the chamber 222 and aperture 214 are in communication. In other embodiments, the body 220 may or may not be attached to the transducer housing 240 prior to attaching to the base 210. The transducer housing 240 is connected to the body 220, aligning the housing aperture 243 with the chamber 222. A retention clip 244 may be temporarily reversibly deformed by a user to allow the clip insert 245 to slide into the retention clip receiver 224, releasing the clip 244 when the insert 245 and receiver 224 are aligned. The insert 245 and receiver 224 holding the body 220 and housing 240 statically together, aided by the additional contours 228 of the body. The transducer housing 240 may or may not contain the transducer 242 therein prior to attachment to the body 220. In any case, the device 200 may be entirely or partially assembled with the base plate 210 prior to attachment to a subject.

The transducer 242 may have been attached to the housing 240 at any point during the above-described assembly process. Once assembled, the transducer 242 may be selectively activated for limited periods of time to avoid heating the tissue 5 via excess acoustic stimulation. In one exemplary embodiment, the transducer 242 may be activated for periods of 5 minutes, with 5-minute rest periods between activations. This may continue for a period of 15 minutes to complete a treatment cycle. As with the other embodiment, treatment cycles using the annular transducer 242 may be of longer or shorter activation and rest periods or total overall treatment time. The recording sites 14 of the implant 10 are targeted during activation, ideally being at a focal point of the acoustic field 260. During activation, the recording sites 14 may cease collecting data, as the acoustic stimulation may introduce artifacts into data output. In some embodiments, the device 200 may be used and modulated therapeutically by substantially the same methods as the disc-shaped transducer embodiment 100, as described in more detail above. The device 200 may be modulated by changing operative parameters such as but not limited to frequency, voltage, temperature, transducer geometry, duty cycle, pulse duration, or I_{SPTA}.

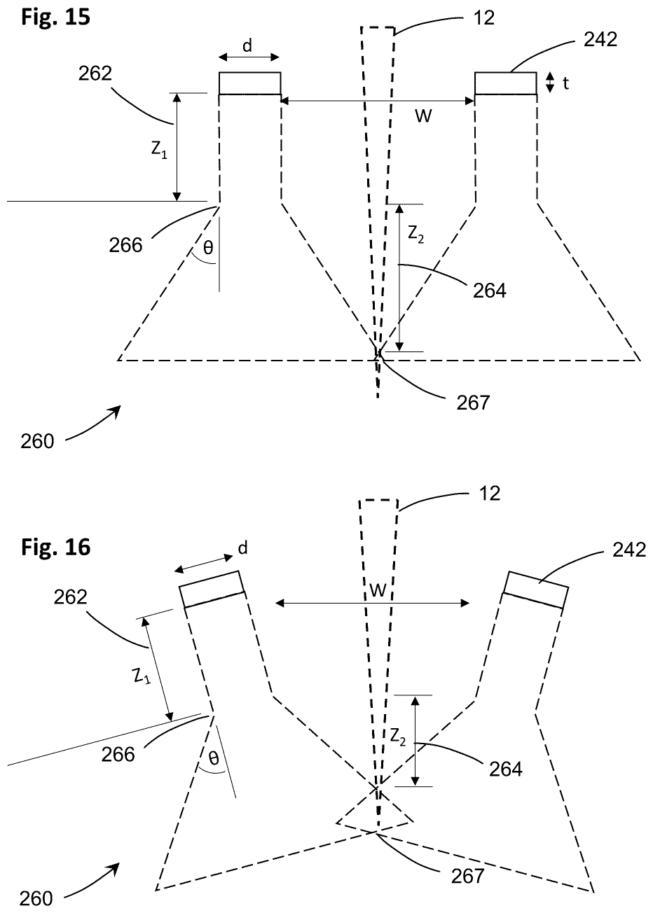

The annular transducer 242 creates a slightly different acoustic field 260 as compared to a disc transducer 142. For instance, taking a longitudinal cross section of the device 200 and annular transducer 242, as shown in FIGS. 15-17, and each side of the transducer 242 may be considered as a single element transducer, acting analogously to the disc-shaped transducer 142 described above. The acoustic field 260 is defined by a near, or proximal, field 262 located adjacent to the transducer 240 and a far, or distal, field 264 located past a transition point 266, penetrating deeper into target tissue 7. The field 260 is approximately the diameter of the transducer 242 within the near field 262 and diverges past the transition point 266 to have increasingly greater diameter than the transducer 240. This divergence from the transition point 266 in the far field 264 is defined by a divergence angle, shown as θ in FIGS. 15-17. The ultrasonic field 260 produced by the transducer may be altered by a variety of factors, including but not limited to the geometry of the transducer, frequency of vibration, thickness of the transducer, acoustic lens application focusing the stimulation, concentric annular piezoelectric elements being selectively excited, and by other factors known in the art.

In some embodiments, the dimensions of the ultrasonic field 260 may be described by a series of equations. Where Z₁ is the length of the near field 262 from the transducer to the transition point 266 and Z₂ is the distance that the far field 264 extends from the transition point 266 to a convergence point 267 of the fields 260 produced by opposing sides of the annular transducer 242:

$${Z}_{1} = \frac{{d}^{2}}{4 λ} \qquad ( 2 )$$

$${Z}_{2} = \frac{W}{2 \cdot tan ( {sin}^{- 1} ( 1.22 λ / d ) )} \qquad ( 3 )$$

where λ=c/f (“d” being the diameter of each side of the annular transducer, taken at a longitudinal cross-section; “c” being sound velocity in tissue, approximately 1,540 m/s; “f” being the frequency of the ultrasound; “W” being the space between opposite sides of the annular transducer 142, measured from the innermost surface thereof). The distance from the transition point 266 to a convergence point 267 is given by Z₂. This convergence point 267 may define an optimal placement point for a given electrode 12. By way of example and without limiting the disclosure herein, with values f=1.1 MHz, d=2 mm, and W=10 mm, Z₁ would equal 0.71 mm and Z₂ would equal 3.04 mm. Any of these factors may be changed to change the field 260 produced by a given transducer 242. Where d=3 mm and all other parameters remain, Z₁ would equal 1.61 mm and Z₂ would equal 7.22 mm, elongating the field 260 with an increase in “d.”

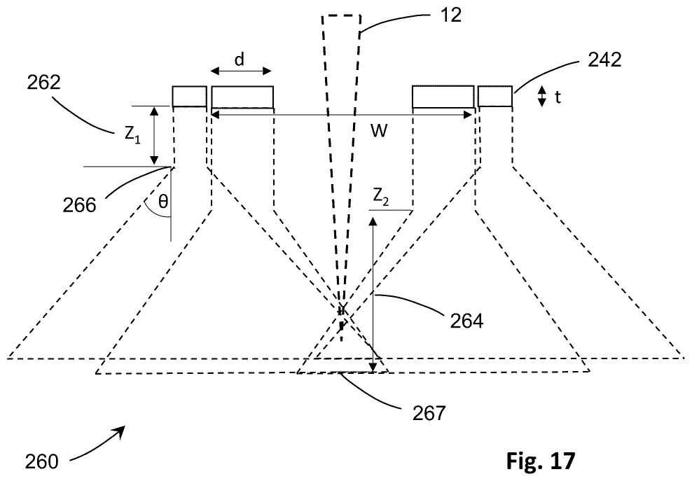

The distance to a convergence point 267, or Z₂, may also be altered by using an annular transducer 242 which has a face at an oblique angle relative to the longitudinal axis 208 of the device 200, as shown in FIG. 16. The fields 260 shown in FIG. 16 may also be produced through the use of a modifier, changing the angle of the field 260. Modifiers may include but are not limited to a lens or wedge. Similarly, as shown in FIG. 17, concentric annular transducer elements 242 may be used in tandem to create overlapping fields 260 with different convergence points 267 which may be optimal for targeting certain electrodes 12.

### Example

To evaluate the effects of low-intensity ultrasound stimulation on long-term neural electrode performance in cortical tissue, an in vivo model was used. Adult subjects (N=10 Sprague Dawley) were implanted with sterile, fixed microelectrode arrays (NeuroNexus, 16 channel 4×4 silicon shanks, 100 μm shank spacing, 125 μm site spacing). Electrode probes were oriented at 45° from horizontal and inserted into cortical layers II/III of the motor or somatosensory cortex using an automated Microdrive to 1.2 mm depth. Subjects were randomly assigned to Stimulation (n=5) or Sham (n=5) treatment groups. During each LIPUS stimulation session, a total of 15 minutes of stimulation was delivered in a periodic fashion to mitigate risk of tissue heating; 5 min ON, 5 min OFF, 5 min ON, 5 min OFF, and 5 min ON. LIPUS and neural recording sessions were conducted daily for days 1-7 post-op and bi-weekly thereafter with subjects lightly anesthetized (0.5-2.0% isoflurane, inhalation) during testing. Electrode impedance measurements and neural signal acquisition (NeuroNexus SmartBox Pro) were taken prior to each LIPUS stimulation session. After six (6) weeks of LIPUS, subjects underwent transcardial perfusion (PBS, followed by 4% paraformaldehyde), and brains were post-fixed, processed and stained for immunohistochemical markers.

The results of these experiments are shown in FIGS. 18A-18B. Specifically, there were insertion-related breakages to the electrode shanks in 2 subjects, leaving n=4 subjects in each treatment group. There were no significant differences in impedance between Control and LIPUS treated groups. Electrophysiology signals were collected via the Allego software package (NeuroNexus) and exported for signal conditioning and spike sorting (SpikeInterface). For each subject, data recorded 5 minutes after the conclusion of LIPUS (or SHAM/Control) treatment was included in this analysis; there were 18 recording days per subject. Interestingly, subjects in the LIPUS Stimulation group demonstrated a significant increase in the percentage of channels that remained active (or had detectible units) after the first week and throughout the rest of the 6-week duration (p<0.0001); ˜40% of channels had ≥1 unit in the LIPUS cohort, while <20% of channels had detectible units in Control subjects. The channels that remained active also maintained a higher signal-to-noise ratio (SNR) over the same time period (p<0.001)

These data show improved electrode longevity using the device and method described herein. Specifically, implanted electrodes treated with the device and method of the present invention showed more active recording channels with better signal-to-noise ratio for the duration of the experiments. In other words, more information was able to be recorded from more neurons for a longer period of time from the electrodes subjected to the treatment described herein than those that were not. This corresponds to a clinically relevant output of a decreased foreign body response (FBR) for the implanted neural devices.

Since many modifications, variations and changes in detail can be made to the described preferred embodiments, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method of reducing foreign body response in a subject from an electrode implanted in neural tissue at a target site, said method comprising:
a) positioning a device having a body with an aperture, an ultrasonic transducer, and acoustic coupling medium retained within the body between and in communication with the ultrasonic transducer and the aperture such that the aperture and acoustic coupling medium are in contact with the neural tissue in proximity to the target site;
b) generating acoustic vibrations by activating said transducer for a predetermined period of time;
c) transmitting said acoustic vibrations to the target site; and
d) applying said acoustic vibrations to the neural tissue at the target site sufficient to reduce immune system foreign body response in the subject where the electrode contacts the neural tissue at the target site.

2. The method as recited in claim 1, wherein said acoustic vibrations are in the ultrasonic frequency range.

3. The method as recited in claim 1, wherein said acoustic vibrations are pulsed, having a duration in the range of about 5 to 200 milliseconds.

4. The method as recited in claim 1, wherein applying said acoustic vibrations to the target site includes applying at least one of a frequency and intensity sufficient to stimulate release of at least one endogenous neurotrophic factor in the target tissue.

5. The method as recited in claim 1, wherein activating said transducer comprises operating said transducer at a frequency in the range of about 200 kHz to 5 MHz, a spatial peak temporal average intensity in the range of about 0.01 to 2.5 W/cm², voltage in the range of about 100 to 600 V, and duty cycle percentage in the range of about 0.5% to 20%.

6. The method as recited in claim 5, wherein activating said transducer comprises operating said transducer at a frequency in the range of about 1.0 to 2.2 MHz, a spatial peak temporal average intensity in the range of about 0.1 to 2.2 W/cm², voltage in the range of about 200 to 280 V, and duty cycle percentage in the range of about 2% to 10%.

7. The method as recited in claim 6, wherein activating said transducer comprises operating said transducer at a frequency of about 1.13 MHz, a spatial peak temporal average intensity of about 0.5 W/cm², voltage of about 280 V, and duty cycle percentage of about 4.2%.

8. The method as recited in claim 1, wherein activating said transducer for a predetermined period of time comprises (i) turning said transducer on for a time in the range about 1 to 15 minutes, (ii) turning said transducer off for a time in the range about 1 to 15 minutes, and (iii) repeating steps (i) and (ii) from 2 to 10 times.

9. The method as recited in claim 8, wherein activating said transducer for said predetermined period of time comprises turning said transducer on for 5 minutes, then off for 5 minutes, then on for 5 minutes for a total treatment time of 15 minutes.

10. The method as recited in claim 8, further comprising repeating steps (b) through (d) once every day during the week following implantation of the electrode in the target tissue.

11. The method as recited in claim 10, further comprising repeating steps (b) through (d) one of: (i) once every other day during the second week following implantation of the electrode in the target tissue, and (ii) once every three days during the second week following implantation of the electrode in the target tissue.

12. The method as recited in claim 1, wherein applying said acoustic vibrations comprises creating an acoustic field of said acoustic vibrations at the target site, said acoustic field surrounding at least a portion of the electrode implanted in the target tissue.

13. The method as recited in claim 12, wherein said acoustic field comprises a near field and a far field separated by a transition point, said far field having a wider diameter than said near field; and at least one of (i) said near field and (ii) said far field surrounding at least a portion of the electrode implanted in the target tissue.

14. The method as recited in claim 12, further comprising creating overlapping acoustic fields of said acoustic vibrations at the target site, at least one of (i) the overlapping portion of said acoustic fields and (ii) a space between said overlapping acoustic fields surrounding at least a portion of the electrode implanted in the target tissue.

15. The method as recited in claim 12, further comprising modulating said acoustic field by changing one of: (i) a frequency of said acoustic vibrations, and (ii) a diameter of said transducer.
